(19) United States
(12) Patent Application Publication
(10) Pub. No.: US 2008/0063610 A1
(43) Pub. Date: Mar. 13, 2008

(54) Use of Beta-Homocyclocitral as an Agent Against Bad Breath

(75) Inventors: Jurgen RABENHORST, Hoxter (DE); Arnold MACHINEK, Holzminden (DE); Steffen SONNENBERG, Markkleeberg (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/575,827

(22) PCT Filed: Sep. 20, 2005
(86) PCT No.: PCT/EP05/54706

§ 371 Date: Mar. 22, 2007

Related U.S. Application Data
(60) Provisional application No. 60/613,024, filed on Sep. 24, 2004.

(51) Int. Cl. A61K 8/31

(52) U.S. Cl. 424/49

(57) ABSTRACT

The use of beta-homocyclocitral is described as an agent (i) to inhibit and/or prevent the growth of and/or to destroy micro-organisms causing bad breath and/or (ii) to combat bad breath.

The invention concerns the use of beta-homocyclocitral as an agent against bad breath, together with corresponding processes. The invention also concerns oral hygiene products containing beta-homocyclocitral.

The healthy human mucosa of the oral and pharyngeal cavity and the solid tooth substance are populated with large numbers of non-pathogenic micro-organisms. This so-called microflora in the oral cavity is not only harmless but also provides important protection against opportunistic or pathogenic bacteria.

A substantial problem of oral hygiene is bad breath, also known as halitosis or foetor ex ore. This odour is formed by the breakdown of food residues and dead cells in the mucosa by micro-organisms. Infestation with gram-positive and gram-negative bacteria and/or mycobionts causes bad breath. Causes cited in the literature are above all anaerobic gram-negative bacteria (e.g. Bad Breath—A multidisciplinary Approach. Eds: D. van Steenberghe, M. Rosenberg, Leuven University Press, Leuven 1996; 111-121). Since social contacts are often impaired by bad breath, those affected by it have a strong interest in remedying or preventing it.

Gram-negative bacteria come for example from the Bacteroides, Fusobacterium, Haemophilus, Neisseria, Porphyromonas, Prevotella, Treponema and Veillonella species.

Gram-positive bacteria are for example representatives from the Actinomyces, Eubacterium, Lactobacillus, Staphylococcus, Stomatococcus and Streptococcus species.

Representatives of mycobionts include, for example, yeasts (Protoascomycetes) and moulds (Plectomycetes).

A distinction must be made here between halitosis and pathological stomatitis (Stomatitis ulcerosa). Whereas halitosis is a normal feature of human breath and so is merely a cosmetic problem, stomatitis is a disease. It is associated with a characteristic, extremely unpleasant odour in the breath.

The object of the present invention was to find substances and agents which are effective against bad breath or against the micro-organisms involved in its formation.

beta-Homocyclocitral (2,6,6-trimethyl-1-cyclohexene-1-acetaldehyde; CAS No. 472-66-2, FEMA No. 3474) is known per se and can be represented by the following formula:

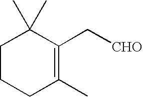

The odour is usually described as camphoraceous, woody, oily, fruity and also herbal.

Extracts and distillates obtainable from the leaves of sugar cane (Saccharum officinarum) together with some aromatic substances contained therein are described in EP 0 941 671. beta-Homocyclocitral was found as a trace component in an extract or distillate. EP 0 941 671 also describes the intensification of the sweetness and the (total) reduction of the bitterness of an edible preparation, for example a diet soft drink, using beta-homocyclocitral in concentrations in the range from 1 to 20 ppb.

The present invention primarily concerns the use of beta-homocyclocitral as an agent (i) to inhibit and/or prevent the growth of and/or to destroy micro-organisms causing bad breath and/or (ii) to combat bad breath.

The present invention also concerns a process to inhibit and/or to prevent the growth of and/or to destroy micro-organisms causing bad breath, with the following step:

Bringing micro-organisms causing bad breath into contact with a quantity of beta-homocyclocitral which is antimicrobially effective against these micro-organisms.

The invention also concerns a process for combating bad breath, with the following step:

Introducing a quantity of beta-homocyclocitral which is antimicrobially effective against micro-organisms causing bad breath into the oral cavity and/or pharyngeal cavity.

A further aspect of the invention concerns an oral hygiene product containing beta-homocyclocitral in a quantity of at least 0.001 wt. %, relative to the total weight of the product.

Other advantageous embodiments of the invention can be seen from the claims, the description below and the examples.

In the present text, oral hygiene products are understood to be the formulations familiar to the person skilled in the art for cleaning and caring for the oral cavity and the pharyngeal cavity and for freshening the breath. Known and common oral hygiene formulations are creams, gels, pastes, foams, emulsions, suspensions, aerosols, sprays and also capsules, granules, pastilles, tablets, sweets or chewing gums, wherein this list of pharmaceutical forms and possible uses is not intended to be limiting. Such formulations serve to clean and care for the tooth substance and oral cavity and to freshen the breath.

The beta-homocyclocitral for use according to the invention is a compound which can be incorporated largely universally into a very wide range of pharmaceutical forms of oral hygiene products, without having to be predefined for one or a few specific pharmaceutical forms, in other words beta-homocyclocitral is compatible with many conventional cosmetic auxiliary substances and additives.

It has also been found that beta-homocyclocitral can both effectively reduce or eliminate bad breath and also prevent its formation, i.e. can also act preventively.

It has further been found that the oral hygiene products according to the invention are effective for a relatively long period, in the order of at least three hours, without any perceptible reduction in their effect.

beta-Homocyclocitral effectively combats bad breath, without significantly damaging the physiological flora of the oral cavity and pharyngeal cavity.

The prior art provided no mention of the use according to the invention of beta-homocyclocitral as an agent to reduce or eliminate bad breath or to prevent the formation of bad breath.

It was found that beta-homocyclocitral can wholly or partly prevent the growth in the oral cavity and pharyngeal cavity of gram-positive and gram-negative bacteria and/or mycobionts which are responsible for bad breath.

It was found that beta-homocyclocitral can wholly or partly prevent the formation of components forming bad breath.

In particular, beta-homocyclocitral is able to inhibit and/or prevent the growth of micro-organisms causing bad breath selected from the group comprising: Eubacterium, Fusobacterium, Haemophilus, Neisseria, Porphyromonas, Prevotella, Treponema and Veillonella species, in particular Fusobacterium nucleatum, Porphyromonas endodontalis, Porphyromonas gingivalis, Prevotella intermedia, Prevotella loeschii and Treponema denticola. In addition, beta-homocyclocitral inhibits and/or prevents the growth of Streptococcus mutans, which plays a part with regard to plaque and plaque formation.

beta-Homocyclocitral is characterised in particular by a very good action against anaerobic and microaerophilic bacteria.

It was also astonishing that beta-homocyclocitral is particularly effective against the especially pronounced morning bad breath which is typically discerned in the morning after getting up.

It has been found in addition that beta-homocyclocitral can wholly or partly prevent the deterioration of oral hygiene products by infestation with gram-positive and gram-negative bacteria and/or mycobionts if it is added to these products in an effective quantity. beta-Homocyclocitral can thus also act as a preservative at the same time.

beta-Homocyclocitral is preferably used according to the invention in oral hygiene products in a quantity in the range from 0.001 (10 ppm) to 10.0 wt. %, in particular with a content of 0.005 (50 ppm) to 5.0 wt. %, particularly preferably with a content of 0.01 (100 ppm) to 4.0 wt. %, and most particularly preferably 0.025 (250 ppm) to 3.0 wt. %, relative to the total weight of the product.

Oral hygiene products containing beta-homocyclocitral in the specified quantities are preferred wherein the beta-homocyclocitral is not used as a constituent of an extract or distillate of sugar cane leaves, as described for example in EP 0 941 671.

It is advantageous to buffer the oral hygiene products according to the invention. A pH range of 3.5 to 10.0 is advantageous.

beta-Homocyclocitral can be easily incorporated into common oral hygiene formulations for oral hygiene products. Preferred oral hygiene products are, for example, tooth creams, toothpastes, tooth gels, mouthwashes, mouth rinses, gargle liquids and mouth or breath sprays, as well as lozenges, pastilles, sweets, chewing gums, chew sweets and dental care chewing gums.

It is also possible and usually preferable to combine beta-homocyclocitral with other ingredients, for example with other antimicrobially active substances, aromatic substances, flavourings and/or auxiliary substances.

The oral hygiene products according to the invention can contain auxiliary substances such as are conventionally used in such preparations, for example preservatives, abrasives, antibacterial agents, anti-inflammatory agents, irritation-preventing agents, irritation-inhibiting agents, antimicrobial agents, antioxidants, astringents, antiseptic agents, antistatics, binders, buffers, support materials, chelating agents, cell stimulants, cleansing agents, conditioning agents, surface-active substances, deodorising agents, softeners, bactericides, emulsifiers, enzymes, ethereal oils, film formers, fixers, foaming agents, foam stabilisers, substances to prevent foaming, foam boosters, gelling agents, gel-forming agents, moisture-releasing agents, moisturising substances, moisture-retaining substances, bleaching agents, optical brighteners, dirt-repelling agents, friction-reducing agents, lubricants, opacifiers, concealing agents, brighteners, polymers, powders, proteins, polishing agents, silicones, skin-calming agents, skin-cleansing agents, skin care agents, skin-healing agents, cooling agents, skin-cooling agents, warming agents, skin-warming agents, stabilisers, suspending agents, thickeners, vitamins, oils, waxes, fats, phospholipids, saturated fatty acids, mono- or polyunsaturated fatty acids, α-hydroxy acids, polyhydroxy fatty acids, liquefiers, dyes, colour-protecting agents, pigments, aromas, flavourings, perfumes or other conventional constituents of a cosmetic or oral hygiene formulation, such as alcohols, polyols, electrolytes, organic solvents, sweeteners, sugar substitutes, silicas, calcium carbonate, calcium hydrogen phosphate, aluminium oxide, fluorides, zinc, tin, potassium, sodium and strontium salts, pyrophosphates, hydrogen peroxide, hydroxyapatites.

If the (oral hygiene) preparation is a solution or lotion, the following can be used as solvents, for example: water or aqueous solutions, oils, such as triglycerides of capric or caprylic acid, or alcohols, diols or polyols having a low C number and ethers thereof, preferably ethanol, isopropanol, propylene glycol, glycerol, ethylene glycol. Mixtures of the aforementioned solvents can naturally also be used.

Examples of flavourings or aromas which can form part of an oral hygiene product according to the invention in addition to beta-homocyclocitral can be found for example in K. Bauer, D. Garbe, H. Surburg, Common Fragrance and Flavor Materials, 4^{th} ed., Wiley-VCH, Weinheim 2001 or in S. Arctander, Perfume and Flavor Chemicals, Vol. I and 11, Montclair, N.J., 1969, self-published.

Examples which may be mentioned of natural aromas which can form part of an oral hygiene product according to the invention in addition to beta-homocyclocitral are: peppermint oils, spearmint oils, mentha arvensis oils, aniseed oils, clove oils, citrus oils, camphor oils, cinnamon oils, cinnamon bark oils, wintergreen oils, eucalyptus oils, eucalyptus citriodora oils, fennel oils, ginger oils, camomile oils, caraway oils, citronella oils, limette oils, orange oils, bergamot oils, grapefruit oils, mandarin oil, rose oils, geranium oils, sage oils, parsley seed oils, yarrow oils, star anise oils, basil oils, bitter almond oils, thyme oils, juniper berry oils, rosemary oils, angelica root oils, vanilla extracts, as well as fractions thereof and ingredients isolated therefrom.

Examples which may be mentioned of homogeneous aromatic substances which can form part of an oral hygiene product according to the invention in addition to beta-homocyclocitral are: anethol, menthol, menthone, isomenthone, menthyl acetate, menthyl propionate, menthofuran, mintlactone, eucalyptol (1,8-cineol), limonene, eugenol, eugenol acetate, thymol, pinene, sabinene hydrate, 3-octanol, carvone, gamma-octalactone, gamma-nonalactone, germacrene-D, viridiflorol, 1,3E,5Z-undecatriene, isopulegol, piperitone, 2-butanone, ethyl formate, 3-octyl acetate, isoamyl isovalerianate, hexanol, hexanal, cis-3-hexenol, linalool, alpha-terpineol, cis- and trans-carvyl acetate, p-cymol, damascenone, damascone, rose oxide, fenchol, acetaldehyde diethylacetal, 1-ethoxyethyl acetate, cis-4-heptenal, isobutyraldehyde, isovaleraldehyde, cisjasmone, methyl dihydrojasmonate, anisaldehyde, methyl salicylate, 2′-hydroxypropiophenone, menthyl methyl ether, myrtenyl acetate, 2-phenylethyl alcohol, 2-phenylethyl isobutyrate, 2-phenylethyl isovalerate, cinnamaldehyde, geraniol, nerol. In the case of chiral compounds the aromatic substances can take the form of a race-mate or a single enantiomer or an enantiomer-concentrated mixture.

Advantageous aromas or aromatic substances which can form part of an oral hygiene product according to the invention in addition to beta-homocyclocitral are, for example, aniseed oil, basil oil, bitter almond oil, camphor oil, citronella oil, citrus oils, eucalyptus citriodora oil, eucalyptus oil, camomile oil, spearmint oil, limette oil, mandarin oil, clove oil, orange oil, peppermint oil, sage oil, thyme oil, wintergreen oil, cinnamon oil, cinnamon bark oil, I-menthol, menthone, 1,8-cineol (eucalyptol), carvone, alpha-terpineol, methyl salicylate, 2′-hydroxypropiophenone, menthyl methyl ether.

Compounds having a physiological cooling effect which can form part of an oral hygiene product according to the invention in addition to beta-homocyclocitral are, for example, I-menthol, menthone glycerol acetal, menthyl lactate, substituted menthyl-3-carboxylic acid amides (e.g. menthyl-3-carboylic acid-N-ethylamide), 2-isopropyl-N,2,3-trimethyl butanamide, substituted cyclohexane carboxylic acid amides, 3-menthoxypropane-1,2-diol, 2-hydroxyethyl menthyl carbonate, 2-hydroxypropyl menthyl carbonate, N-acetyl glycine menthyl ester, menthyl hydroxycarboxylic acid esters (e.g. menthyl-3-hydroxybutyrate), monomenthyl succinate 2-mercaptocyclodecanone, menthyl-2-pyrrolidin-5-one carboxylate, 2,3-dihydroxy-p-menthane, 3,3,5-trimethyl cyclohexanone glycerol ketal, 3-menthyl-3,6-di- and tri-oxaalkanoates, 3-menthyl methoxyacetate, icilin, I-menthyl methyl ether. I-Menthol, menthone glycerol acetal, menthyl lactate, menthyl-3-carboxylic acid-N-ethylamide, 3-menthoxypropane-1,2-diol, 2-hydroxyethyl menthyl carbonate, 2-hydroxypropyl menthyl carbonate, monomenthyl succinate, menthyl-2-pyrrolidin-5-one carboxylate, I-menthyl methyl ether are preferred.

Constituents which bring about a sensation of heat, sharpness, itching or prickling on the skin or on the mucous membranes, in particular aromatic substances having a heat-generating effect and/or compounds having a pungent taste (pungent principles), which can form part of an oral hygiene product according to the invention in addition to beta-homocyclocitral are, for example, capsaicin, dihydrocapsaicin, gingerol, paradol, shogaol, piperine, paprika powder, chilli pepper powder, extracts of paprika, extracts of pepper; extracts of chilli pepper; extracts of ginger roots, extracts of Aframomum melegueta, extracts of Spilanthes acmella, extracts of Kaempferia galanga, extracts of Alpinia galanga, carboxylic acid-N-vanillylamides, in particular nonanoic acid-N-vanillylamide, 2-nonenoic acid amides, in particular 2-nonenoic acid-N-isobutylamide, 2-nonenoic acid-N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylene dioxybenzyl alcohol, (4-hydroxy-3-methoxyphenyl)acetic acid amides, in particular (4-hydroxy-3-methoxyphenyl)acetic acid-N-n-octylamide, nicotinaldehyde, methyl nicotinate, propyl nicotinate, 2-butoxyethyl nicotinate, benzyl nicotinate, 1-acetoxychavicol.

Other constituents which can form part of an oral hygiene product according to the invention in addition to beta-homocyclocitral are, for example, substances to improve oral hygiene, such as dental care and/or refreshing substances, for example. Substances to improve oral hygiene include, for example, substances to combat or prevent plaque, tartar or caries and those to combat or prevent bad breath. Reference is made in this connection to U.S. Pat. No. 5,043,154. Substances which can be cited by way of example are Zn salts, such as Zn citrate, Zn fluoride, Sn salts, such as Sn fluorides, Cu salts, fluorides, e.g. amine fluorides, alkali fluorides such as Na fluoride, alkaline-earth fluorides, ammonium fluoride, phosphates, pyrophosphates, fluorophosphates, such as Na monofluorophosphate, Al mono- and Al difluorophosphate, alpha-ionones, geraniol, thymol, eugenol, isomenthyl acetate, panthenol (provitamin B₅), xylitol, allantoin, niacinamide (vitamin B₃), tocopheryl acetate (vitamin E acetate), poloxamer. An oral hygiene product according to the invention can also contain in addition to beta-homocyclocitral one or more other antimicrobial active ingredients to improve oral hygiene. These active ingredients can be of a hydrophilic, amphoteric or hydrophobic nature. Examples which can be cited are: triclosan, chlorhexidines and salts, peroxides, phenols and salts thereof, domiphen bromide (phenododecinium bromide), bromochlorophene, Zn salts, chlorophylls, Cu salts, Cu gluconate, Cu chlorophyll, sodium lauryl sulfate, quaternary monoammonium salts such as cocoalkyl benzyl dimethyl ammonium chloride or pyridinium salts such as cetyl pyridinium chloride. In addition to individual active ingredients, mixtures of active ingredients or natural extracts or fractions thereof containing active ingredients can be used, such as those obtainable from neem, berberis, fennel, green tea, marigold, camomile, rosemary, thyme, propolis or turmeric, for example.

In addition to beta-homocyclocitral, an oral hygiene product according to the invention can also contain antioxidants, the following being cited here by way of example: carotenoids, carotenes (e.g. α-carotene, β-carotene, lycopene) and derivatives thereof, flavonoids, quercetin, (metal) chelators (e.g. α-hydroxy fatty acids, fatty acids (palmic acids), phytic acid, lactoferrin, EDTA, EGTA), α-hydroxy acids (e.g. citric acid, lactic acid, malic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), ferulic acid and derivatives thereof, butyl hydroxytoluene (BHT), butyl-hydroxyanisole, zinc and derivatives thereof (e.g. ZnO, ZnSO₄), selenium and derivatives thereof (e.g. selenium methionine); ingredients isolated from plants, extracts or fractions thereof, e.g. from tea, green tea, algae, grape seeds, wheat germ, camomile, rosemary, oregano.

In addition to beta-homocyclocitral, an oral hygiene product according to the invention can also contain dyes, colorants or pigments, the following being cited here by way of example: lactoflavin (riboflavin), beta-carotene, riboflavin-5′-phosphate, alpha-carotene, gamma-carotene, cantaxanthin, erythrosine, curcumin, quinoline yellow, yellow orange S, tartrazine yellow, bixin, norbixin (annatto, orlean), capsanthin, capsorubin, lycopene, beta-apo-8′-carotenal, beta-apo-8′-carotenic acid ethyl ester, xantophylls (flavoxanthin, lutein, cryptoxanthin, rubixanthin, violaxanthin, rodoxanthin), fast carmine (carminic acid, cochineal), azorubin, cochineal red A (Ponceau 4 R), beetroot red, betanin, anthocyanins, amaranth, patent blue V, indigotine I (indigo carmine), chlorophylls, copper compounds of chlorophylls, acid brilliant green BS (lissamine green), brilliant black BN, vegetable carbon, titanium dioxide, iron oxides and hydroxides, calcium carbonate, aluminium, silver, gold, pigment rubine BK (lithol rubine BK), methyl violet B. victoria blue R. victoria blue B. acilan brilliant blue FFR (brilliant wool blue FFR), naphthol green B. acilan fast green 10 G (alkali fast green 10 G), ceres yellow GRN, sudan blue 11, ultramarine, phthalocyanine blue, phthalocyanine green, fast acid violet R. Other, naturally obtained extracts (e.g. paprika extract, black carrot extract, red cabbage extract) can also be used for colouring purposes.

The following examples are intended to clarify the present invention, without restricting its scope.

### EXAMPLES

Unless otherwise specified, all figures stated relate to the weight.

In-Vitro Test to Reduce Bad Breath

The test is based on the work of Goldberg and Rosenberg (Production of Oral Malodor in an in vitro System, S. Goldberg and M. Rosenberg, pp.143 -150, in: Bad Breath—A multidisciplinary Approach, Eds: D. van Steenberghe, M. Rosenberg, Leuven University Press, 1996) and has been adapted to improve reproducibility.

A sterile liquid medium inoculated with fresh morning saliva is incubated for a few days at 37° C. and then sniffed by a panel of testers.

An intensive odour characteristic of bad breath has formed. Uninoculated controls have only a faint odour of the medium.

As a control for the tests, Triclosan® (5-chloro-2-(2,4-dichlorophenoxy)phenol) was added to inoculated samples in a concentration of 0.05%. After the incubation period, inoculated samples treated with Triclosan® had the same faint odour of medium as the uninoculated samples.

With the addition of 0.025% beta-homocyclocitral to inoculated samples, no odour—just as with Triclosan®—could be determined after the incubation period. This value also corresponds to the minimum effective concentration. With 0.01%, a changed characteristic odour or a mixed odour was discernible in some cases.

With the addition of 0.05% beta-homocyclocitral at the start of the test, a slight characteristic odour is discernible after the incubation period.

If 0.05% beta-homocyclocitral is added to the inoculated medium just one hour before the sensory assessment, an unpleasant mixed odour of bad breath and a faint beta-homocyclocitral note is observed. This observation proves that the action of beta-homocyclocitral against bad breath is not an olfactory masking/concealing action.

Determination of the Minimum Inhibiting Concentration

5 The minimum inhibiting concentration (MIC) of beta-homocyclocitral was determined in a serial-dilution test against various bacteria. The result is shown in the table below:

| Organism | MIC [ppm] | Type |
|---|---|---|
| Escherichia coli | >1000 | No action up to 1000 ppm |
| Pseudomonas aeruginosa | >1000 | No action up to 1000 ppm |
| Fusobacterium nucleatum | 250 | Bactericidal |
| Prevotella intermedia | 250 | Bactericidal |
| | 125 | Bacteriostatic |
| Streptococcus mutans | 500 | Bactericidal |
| Veilonella parvula | 500 | Bactericidal |
| Porphyromonas gingivalis | 125 | |
| Staphylococcus aureus | 500 | |

A bactericidal action against the halitosis-causing bacteria Fusobacterium nucleatum, Prevotella intermedia and Veilonella parvula was thus demonstrated. In addition, beta-homocyclocitral has a bactericidal action against Streptococcus mutans, which plays a part in plaque and plaque formation, and in the formation of caries.

In a further test in a concentration of 1000 ppm, beta-homocyclocitral showed no significant reduction with regard to the total number of bacteria in the samples of saliva which were examined. Furthermore, in further tests in a concentration of 1000 ppm, beta-homocyclocitral showed no significant reduction with regard to Pseudomonas aruginosa and Escherichia coli.

### Formulation Examples

1. Gel Toothpaste Effective Against Bad Breath

| | I (%) | II (%) | III (%) |
|---|---|---|---|
| Na carboxymethyl cellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccharinate | 0.07 | 0.07 | 0.07 |
| Na fluoride | 0.24 | 0.24 | 0.24 |
| p-Hydroxybenzoic acid (PHB) ethyl | 0.15 | 0.15 | 0.15 |
| ester | | | |
| Flavour | 1.0 | 1.00 | 1.00 |
| beta-Homocyclocitral | 0.025 | 0.06 | 0.10 |
| Abrasive silica | 11.00 | 11.00 | 11.00 |
| Thickening silica | 6.00 | 6.00 | 6.00 |
| Sodium dodecyl sulfate (SDS) | 1.40 | 1.40 | 1.40 |
| Water dist. | To make | To make | To make |
| | 100.00 | 100.00 | 100.00 |

2. Anti-Plaque Toothpaste Effective Against Bad Breath

| | I (%) | II (%) | III (%) |
|---|---|---|---|
| Na carboxymethyl cellulose | 1.00 | 1.00 | 1.00 |
| Glycerine | 12.50 | 12.50 | 12.50 |
| Sorbitol 70%, in water | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphosphoric | 1.00 | 1.00 | 1.00 |
| acid, disodium salt | | | |
| Bromochlorophene | 0.10 | 0.10 | 0.10 |
| Peppermint flavour | 1.10 | 1.10 | 1.10 |
| beta-Homocyclocitral | 0.025 | 0.06 | 0.10 |
| Abrasive silica | 15.00 | 15.00 | 15.00 |
| Thickening silica | 5.00 | 5.00 | 5.00 |
| Sodium dodecyl sulfate (SDS) | 1.50 | 1.50 | 1.50 |
| Water dist. | To make | To make | To make |
| | 100.00 | 100.00 | 100.00 |

3. Anti-Plaque Toothpaste Effective Against Bad Breath

Basis: Silica, alkali diphosphate

| | I (%) | II (%) | III (%) |
|---|---|---|---|
| Carrageenan | 0.90 | 0.90 | 0.90 |
| Glycerine | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccharinate | 0.40 | 0.40 | 0.40 |
| Precipitated silica | 20.00 | 20.00 | 20.00 |
| Titanium dioxide | 1.00 | 1.00 | 1.00 |
| PHB methyl ester | 0.10 | 0.10 | 0.10 |
| Spearmint flavour | 1.10 | 1.10 | 1.10 |
| beta-Homocyclocitral | 0.025 | 0.06 | 0.10 |
| Sodium dodecyl sulfate | 1.30 | 1.30 | 1.30 |
| Water dist. | To make 100.00 | To make | To make |
| | | 100.00 | 100.00 |

4. Toothpaste for Sensitive Teeth Effective Against Bad Breath

| | I (%) | II (%) | III (%) |
|---|---|---|---|
| Na carboxymethyl cellulose | 0.70 | 0.70 | 0.70 |
| Xanthan gum | 0.50 | 0.50 | 0.50 |
| Glycerine | 15.00 | 15.00 | 15.00 |
| Sorbitol 70%, in water | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Flavour | 1.00 | 1.00 | 1.00 |
| beta-Homocyclocitral | 0.025 | 0.06 | 0.10 |
| Ca carbonate | 35.00 | 35.00 | 35.00 |
| Silicon dioxide | 1.00 | 1.00 | 1.00 |
| Sodium dodecyl sulfate | 1.50 | 1.50 | 1.50 |
| (SDS) | | | |
| Water dist. | To make | To make | To make |
| | 100.00 | 100.00 | 100.00 |

5. Toothpaste for Sensitive Teeth Effective Against Bad Breath

| | I (%) | II (%) | III (%) |
|---|---|---|---|
| Hydroxyethyl cellulose | 1.40 | 1.40 | 1.40 |
| Guar gum | 0.60 | 0.60 | 0.60 |
| Glycerine | 18.00 | 18.00 | 18.00 |
| Sorbitol 70%, in water | 12.00 | 12.00 | 12.00 |
| Na saccharinate | 0.35 | 0.35 | 0.35 |
| Dye | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Cinnamon flavour | 1.20 | 1.20 | 1.20 |
| beta-Homocyclocitral | 0.025 | 0.06 | 0.1 |
| Precipitated silica | 15.00 | 15.00 | 15.00 |
| Silicon dioxide | 1.60 | 1.60 | 1.60 |
| Sodium dodecyl sulfate | 1.30 | 1.30 | 1.30 |
| Water dist. | To make | To make | To make |
| | 100.00 | 100.00 | 100.00 |

6. Ready-to-Use Mouthwash with Fluoride and Effective Against Bad Breath

| | I (%) | II (%) | III (%) |
|---|---|---|---|
| Ethanol | 7.00 | 7.00 | 7.00 |
| Glycerine | 12.00 | 12.00 | 12.00 |
| Na fluoride | 0.05 | 0.05 | 0.05 |
| Pluronic F-127 ® (BASF, | 1.40 | 1.40 | 1.40 |
| surface-active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Peppermint flavour | 0.15 | 0.15 | 0.15 |
| beta-Homocyclocitral | 0.01 | 0.02 | 0.03 |
| Dye | 0.01 | 0.01 | 0.01 |
| Water dist. | To make | To make | To make |
| | 100.00 | 100.00 | 100.00 |

7. Concentrated Mouthwash Effective Against Bad Breath

| | I (%) | II (%) | III (%) |
|---|---|---|---|
| Ethanol, 95% | 80.00 | 80.00 | 80.00 |
| Na cyclamate | 0.15 | 0.15 | 0.15 |
| Cinnamon flavour | 3.50 | 3.50 | 3.50 |
| Dye | 0.01 | 0.01 | 0.01 |
| beta-Homocyclocitral | 0.50 | 1.0 | 3.0 |
| Water dist. | To make 100.00 | To make 100.00 | To make |
| | | | 100.00 |

8. Chewing Gum to Prevent Bad Breath

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerine | 0.50 | 0.50 | 0.50 |
| Icing sugar | 60.45 | 60.40 | 60.30 |
| Spearmint flavour | 1.50 | 1.50 | 1.50 |
| beta-Homocyclocitral | 0.05 | 0.10 | 0.20 |

9. Sugar-Free Chewing Gum to Prevent Bad Breath

| | I (%) | II (%) | III (%) |
|---|---|---|---|
| Chewing gum base | 30.00 | 30.00 | 30.00 |
| Sorbitol, powdered | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame K | 0.10 | 0.10 | 0.10 |
| Emulgum/emulsifier | 0.30 | 0.30 | 0.30 |
| Sorbitol 70%, in water | 14.00 | 14.00 | 14.00 |
| Glycerine | 1.00 | 1.00 | 1.00 |
| Cinnamon-menthol flavour | 1.50 | 1.50 | 1.50 |
| beta-Homocyclocitral | 0.05 | 0.10 | 0.20 |

10. Gelatine Capsule for Direct Consumption to Prevent Bad Breath

| | I (%) | II (%) | III (%) |
|---|---|---|---|
| Gelatine shell: | | | |
| Glycerine | 2.014 | 2.014 | 2.014 |
| Gelatine 240 bloom | 7.91 | 7.91 | 7.91 |
| Sucralose | 0.065 | 0.065 | 0.065 |
| Allura Red | 0.006 | 0.006 | 0.006 |
| Brilliant Blue | 0.005 | 0.005 | 0.005 |
| Core composition: | | | |
| Plant oil triglyceride | 80.0 | 75.0 | 60.0 |
| Flavour B | 7.0 | 10.0 | 20.0 |
| beta-Homocyclocitral | 3.0 | 5.0 | 10.0 |

Flavour B had the following composition (figures in wt. %):

0.1% neotame powder, 0.05% aspartame, 29.3% peppermint oil arvensis, 29.3% peppermint piperita oil Willamette, 2.97% sucralose, 2.28% triacetin, 5.4% diethyl tartrate, 12.1% peppermint oil yakima, 0.7% ethanol, 3.36% 2-hydroxyethyl menthyl carbonate, 3.0% 2-hydroxypropyl menthyl carbonate, 0.27% vanillin, 5.5% Dlimonene, 5.67% L-menthyl acetate.

The gelatine capsule suitable for direct consumption had a diameter of 5 mm and the weight ratio of core material to shell material was 90:10. The capsule opened in the mouth in less than 10 seconds and dissolved completely in less than 50 seconds.

1. A method of inhibiting the growth of micro-organisms causing bad breath and/or to combat bad breath by adding thereto a composition beta-homocyclocitral in an amount sufficient to act as an agent (i) to inhibit and/or prevent the growth of and/or to destroy micro-organisms causing bad breath and/or (ii) to combat bad breath.

2. A method according to claim 1, wherein the mirco-organisms causing bad breath are selected from the group comprising; Eubacterium, Fusobacterium, Haemophilus, Neisseria, Porphyromonas, Prevotella, Treponema, and Veilonella species.

3. An oral hygiene product containing beta-homocyclocitral in a quantity of at least 0.001 wt. %, relative to the total weight of the product.

4. An oral hygiene product according to claim 3, containing a quantity of beta-homocyclocitral in the range from 0.001 to 10.0 wt. %, relative to the total weight of the product.

5. A process to inhibit and/or to prevent the growth of and/or to destroy micro-organisms causing bad breath, comprising the following step:
bringing micro-organisms causing bad breath into contact with quantity of beta-homocyclocitral which is antimicrobially effective against these micro-organisms.

6. A process for combating bad breath, comprising the following step:
introducing a quantity of beta-homocyclocitral which is antimicrobially effective against micro-organisms causing bad breath into the oral cavity and/or pharyngeal cavity.
